(19) United States
(12) Patent Application Publication
(10) Pub. No.: US 2010/0267824 A1
(43) Pub. Date: Oct. 21, 2010

(54) STABLE OXALIPLATIN COMPOSITION FOR PARENTERAL ADMINISTRATION

(75) Inventors: Ashish Sehgal, Ahmedabad (IN); Bhavesh Vallabhabhai Patel, Ahmedabad (IN); Jayanta Kumar Mandal, Ahmedabad (IN)

Correspondence Address: HAMRE, SCHUMANN, MUELLER & LARSON, P.C., P.O. BOX 2902, MINNEAPOLIS, MN, 55402-0902, US

(73) Assignee: INTAS PHARMACEUTICALS LIMITED, Ahmedabad, Gujarat (IN)

(21) Appl. No.: 12/742,378

(22) PCT Filed: Nov. 11, 2008
(86) PCT No.: PCT/IN2008/000765

§ 371 Date: May 11, 2010

(30) Foreign Application Priority Data
Nov. 12, 2007 (IN) 2237/MUM/2007

(51) Int. Cl. A61K 31/282; A61P 35/00

(52) U.S. Cl. 514/492

(57) ABSTRACT

The present invention relates to a stable parenteral composition of oxaliplatin having pH range in between 3 to 4.5, which comprises of a solution of oxaliplatin in water wherein the said pH is attained by sparging of carbon dioxide in the composition. Further, a method for the preparation of oxaliplatin composition of the present invention is also disclosed.

### FIELD OF THE INVENTION

The present invention relates to stable composition of oxaliplatin in aqueous solution of carbohydrate, wherein stability of the composition is attained by sparging of carbon dioxide.

### BACKGROUND

Oxaliplatin has been adapted rapidly due to its in vitro and in vivo anti tumoral activity and good clinical tolerance with low toxicity. Oxaliplatin is a potent compound for the treatment of various kind of cancers particularly, those of the colon, of the ovaries, of the upper respiratory tract and also epidermoid cancers.

U.S. Pat. No. 5,716,988 discloses aqueous solution of oxaliplatin, having pH range 4.5 to 6, for parenteral administration which is free of any acidic or alkaline agent, buffer or other additives.

While formulating composition of oxaliplatin in water, without use of any additives, even though the pH is achieved, it is observed that composition remains unstable in water due to formation of impurities, which results in to instability of platinum complex and thereby destruction of the complex. Hence it is crucial to use additives to provide stable oxaliplatin solution for parenteral administration.

To avoid destruction of platinum complex and achieve better stability of aqueous oxaliplatin solutions, alternatively an acid is to be added to the platinum complex in an aqueous solution to reduce the hydroxide anion concentration. Use of various acids has been found in the following patents to surmount instability of aqueous solution of oxaliplatin.

U.S. Pat. No. 6,306,902 discloses oxaliplatin composition comprising a therapeutically effective amount of oxaliplatin, an effective stabilizing amount of oxalic acid or its alkali metal salt as a buffering agent and a pharmaceutically acceptable carrier.

U.S. Pat. No. 6,476,068 discloses oxaliplatin composition comprising a therapeutically effective amount of oxaliplatin, an effective stabilizing amount of lactic acid or its salt and a pharmaceutically acceptable carrier.

US 20060063833 discloses oxaliplatin composition comprising a therapeutically effective amount of oxaliplatin in water with an acid selected from the group consisting of phosphoric acid, sulfuric acid, methane sulfonic acid, ethane sulfonic acid, para-toluene sulfonic acid, and mixtures thereof and carbohydrates such as lactose, glucose, maltose, fructose, galactose, and/or dextrans. Further this patent also rundown the use of hydrochloric acid and sodium chloride as both additives cause the oxaliplatin complex to degrade undesirably by substituting chloride ions for the unstable ligands.

US 20060264501 discloses oxaliplatin composition comprising a therapeutically effective amount of oxaliplatin in water with an acid selected from the group consisting of group consisting of citric acid, maleic acid, saccharic acid, succinic acid, malic acid, tartaric acid and mixtures thereof.

However there is peril that the anion resulting from an acid may cause the platinum complex to decompose or change and further increase the rate of formation of secondary degradation products by using an acid. Further it is also observed that the acids are capable to form water insoluble salt crystals with calcium and magnesium cations, which can be found in blood.

To avoid above said disadvantages, desirable objective of the present invention is to enhance the stability and also that the composition can be suitably kept for a prolonged period.

The present invention meets these objectives by providing oxaliplatin compositions, with superior stability properties compared with the above-identified known preparations. It has been found that the introduction of carbon dioxide gas by sparging in an aqueous solution of oxaliplatin reduces the pH and serves as a novel method of making a composition with an improved stability versus above-identified known preparations.

The term “Stability studies” herein refers to accelerated stability studies performed on the injectable oxaliplatin composition of present invention.

The term “Impurities” herein refers to the degradation products of oxaliplatin obtained either due to hydrolysis or oxidation of oxaliplatin. Impurities of oxaliplatin obtained according to European Pharmacopoeia are Impurity A, Impurity B, Impurity C, Impurity E and other impurities.

The term “Impurity A” herein refers to ethanedioic acid (oxalic acid). Impurity A of oxaliplatin is the degradation product of oxaliplatin formed due to hydrolysis of oxaliplatin.

The term “Impurity B” herein refers to (SP-4-2)-diaqua[(1R,2R)-cyclohexane-1,2-diamine-κN, κN′] platinum (diaquodiaminocyclohexaneplatinum). Impurity B of oxaliplatin is the degradation product of oxaliplatin formed due to hydrolysis of oxaliplatin.

The term “Impurity C” herein refers to (OC-6-33)-[(1R,2R)-cyclohexane-1,2-diamine-κN, κN'][ethanedioato(-2-), κO)dihydroxyplatinum. Impurity C of oxaliplatin is the degradation product of oxaliplatin due to oxidation of oxaliplatin.

The term “Impurity E” herein refers to (SP-4-2)-di-g-oxobis[(1R,2R)-cyclohexane-1,2-diamine-κN, κN′]diplatinum (diaquodiamino cyclohexane platinum dimer).

The term “other impurities” herein refers to other non-significant unidentified impurities of oxaliplatin formed in the oxaliplatin composition of the present invention.

The term “sparging” or “sparging technique” herein refers to bubbling of carbon dioxide gas through bulk solution.

### OBJECTS OF THE INVENTION

The main object of the invention is to provide stable oxaliplatin composition and prevent the decomposition or change in platinum complex and there by reducing the rate of formation of secondary degradation products.

Another object of the invention is to sparge carbon dioxide in composition to get stable oxaliplatin composition.

Another object of the invention is to provide composition, having pH 3 to 4.5 suitable for parenteral administration with enhanced stability that can be suitably kept for a prolonged period.

Still another object of the invention is the process of preparation of stable oxaliplatin composition with sparging of carbon dioxide.

### SUMMARY OF THE INVENTION

The present invention is directed towards a stable aqueous oxaliplatin composition having pH 3 to 4.5 wherein the said pH is achieved by sparging of carbon dioxide.

Another embodiment of the invention that directed towards a stable oxaliplatin composition is aqueous solution of carbohydrate in addition to sparging of carbon dioxide, wherein pharmaceutically acceptable carbohydrates are selected from lactose mono hydrate, glucose, maltose, fructose, trehalose, sucrose, galactose, dextran or mixtures thereof, which improves solubility of oxaliplatin.

### DETAILED DESCRIPTION OF THE INVENTION

It has been discovered by the inventors of the present invention that aqueous solution of oxaliplatin composition possessing long storage life, stability, acceptable levels of degradation products and impurities formed during storage of the aqueous oxaliplatin composition could be obtained by sparging of carbon dioxide to the aqueous oxaliplatin composition and attaining a pH of 3 to 4.5.

Further, sparging of carbon dioxide and attaining the pH in between 3 to 4.5 of the aqueous oxaliplatin composition of the present invention does not reduce the concentration of oxaliplatin less than 98% of the initial oxaliplatin concentration and leads to minimal formation of oxaliplatin degradation products or impurities.

The primary object of the present invention is to provide stable oxaliplatin composition by dissolving oxaliplatin, ranging from 1 mg/ml to 7 mg/ml, preferably 5 mg/ml, in aqueous solution wherein sparging of carbon dioxide is made to attain a pH of 3 to 4.5.

Another embodiment of the present invention is directed towards the stability of aqueous oxaliplatin composition having pH 3 to 4.5, wherein the pH in between 3 to 4.5 of the oxaliplatin composition reduces the hydrolysis of oxaliplatin and helps in stabilizing impurity A and impurity B of oxaliplatin. As per the present invention, sparging carbon dioxide in the aqueous oxaliplatin composition helps in removal of dissolved oxygen and replacing it by carbon dioxide, thereby preventing the formation of impurity C of oxaliplatin that is formed due to oxidation of oxaliplatin.

Another embodiment of the present invention is directed towards the stability of the aqueous oxaliplatin composition having pH 3 to 4.5, wherein the oxaliplatin content in the aqueous oxaliplatin composition will not reduce less than 98% of the initial oxaliplatin concentration and the oxaliplatin solution remains clear, colorless and free from particulates after storage for a pharmaceutically acceptable duration.

This primary invention is formulated by the process as per the following steps:

Step 1: Take carbon dioxide sparged water for injection, 90% of proposed batch size at 50-60° C. and add Oxaliplatin in the solution and stir till complete dissolution and thereby clear solution is obtained.

Step 2: Sparge carbon dioxide in the solution of step 1 till the pH range is attained in between 3 to 4.5.

Step 3: Cool down the solution to 20-25° C. and make up the volume to the proposed batch size with water for injection.

Step 4: Again sparge carbon dioxide gas with gentle stirring till pH is attained in between 3 to 4.5.

Further the present invention also provides stable composition in which oxaliplatin is dissolved in aqueous solution of carbohydrate followed by sparging of carbon dioxide wherein amount of oxaliplatin is ranging from 1 mg/ml to 7 mg/ml, preferably 5 mg/ml.

The pharmaceutically acceptable carbohydrates, used in the present invention, may be selected from the group comprising, but not limited to, lactose mono hydrate, sucrose, glucose, maltose, fructose, trehalose, galactose, dextran and the like or mixtures thereof, wherein the said carbohydrates are in the range of 10 to 70 mg/mL, preferably 40 to 50 mg/mL, more preferably 45 mg/mL.

The present invention is formulated by process as per the following steps:

Step 1: Take carbon dioxide sparged water for injection, 90% of proposed batch size at 50-60° C. and add oxaliplatin in it, stir till clear solution is obtained.

Step 2: Add lactose monohydrate in the solution of step 1 and stir till complete dissolution and thereby clear solution is obtained.

Step 3: Sparge carbon dioxide in the solution of step 2 Step 4: Cool down the solution to 20-25° C. and make up the volume to the proposed batch size with water for injection.

Step 5: Again sparge carbon dioxide gas with gentle stirring.

Throughout this specification and the appended claims it is to be understood that the words “comprise” and include” and variations such as “comprises”, “comprising”, “includes”, “including” are to be interpreted inclusively, unless the context requires otherwise. That is, the use of these words may imply the inclusion of an element or elements not specifically recited.

### EXAMPLES

The present invention has been described by way of example only, and it is to be recognized that modifications thereto which fall within the scope and spirit of the appended claims, and which would be obvious to a skilled person based upon the disclosure herein, are also considered to be included within the invention.

Methods for preparing the composition of the present invention could be represented by the following examples

The above said invention of aqueous solution of oxaliplatin can be illustrated by but not limited to following examples.

### Example 1

5 mg of oxaliplatin is added to carbon dioxide sparged water for injection, 90% of the proposed batch size at 50-60° C. with constant stirring to get clear solution. 45 mg of lactose monohydrate is added to this solution followed by sparging of carbon dioxide till clear solution is obtained. Solution is cooled to 20-25° C. Make up the volume with water for injection and sparge further with carbon dioxide for 60 minutes with stirring.

### Stability Studies:

| | | Accelerated stability (40° C. ± |
|---|---|---|
| | | 2° C. & 75% ± 5% RH) |
| Parameters | Initial | 6 Month |
| pH | 4.2 | 4.2 |
| Assay | 103.3% | 99.8% |

Related substances

| | | |
|---|---|---|
| Impurity A | 0.20% | 0.27% |
| Impurity B | 0.3% | 0.36% |
| Impurity E | ND | ND |
| Impurity C | 0.01% | 0.18% |
| Other impurity | ND | 0.10% |
| Total impurities | 0.5% | 1.03% |

ND—not detected

Results of the stability studies performed for oxaliplatin composition described according to example 1 mention that the pH, assay for oxaliplatin and the amount of impurities formed after an accelerated studies conducted for 6 months were within the acceptable limits.

### Example 2

5 mg of oxaliplatin is added to water for injection, 90% of the proposed batch size at 50-60° C. with constant stirring to get clear solution followed by sparging of carbon dioxide till clear solution is obtained. Solution is cooled to 20-25° C. and the volume is made with water for injection. Sparge further with carbon dioxide for 60 minutes with stirring.

### Stability Studies:

| | | Accelerated stability (40° C. ± 2° C. |
|---|---|---|
| | | & 75% ± 5% RH) |
| Parameters | Initial | 6 Month |
| pH | 4.2 | 4.2 |
| Assay | 98.9 | 98.2 |

Related substances

| | | |
|---|---|---|
| Impurity A | 0.149 | 0.18 |
| Impurity B | 0.26 | 0.19 |
| Impurity E | ND | ND |
| Impurity C | 0.005 | ND |
| Other impurity | 0.023 | 0.12 |
| Total impurities | 0.445 | 0.49 |

ND—not detected

Results of the stability studies performed for oxaliplatin composition described according to example 2 mentions that the pH, assay for oxaliplatin and the amount of impurities formed after an accelerated studies conducted for 6 months were within the acceptable limits.

### Example 3

5 mg of oxaliplatin is added to water for injection, 90% of the proposed batch size at 50-60° C. with constant stirring to get clear solution. 45 mg of lactose monohydrate is added to this solution till a clear solution is obtained. Solution is cooled to 20-25° C. and the volume is made with water for injection.

### Stability Studies:

| | | Accelerated stability | |
|---|---|---|---|
| | | (40° C. ± 2° C. | |
| | | & 75% ± 5% RH) | |
| Parameters | Initial | 1 Month | 6 Month |
| pH | 4.9 | 5.02 | Not performed |
| Assay | 98.1 | 96.7 | Not performed |

Related substances

| | | | |
|---|---|---|---|
| Impurity A | 0.182 | 0.269 | Not performed |
| Impurity B | 0.358 | 0.525 | Not performed |
| Impurity E | ND | ND | Not performed |
| Impurity C | 0.007 | 0.184 | Not performed |
| Other impurity | ND | 0.273 | Not performed |
| Total impurities | 0.547 | 1.265 | Not performed |

ND—not detected

According to the stability studies performed for oxaliplatin composition described according to example 3 (non-sparged oxaliplatin composition), the amount of impurities formed after an accelerated studies conducted for a period of one month revealed an increased amount of impurities as compared to the levels described in example 1 or example 2. Further, the stability studies for example 3 after a period of 1 month were terminated because of high levels of impurities in the composition.

The results obtained from the stability studies performed on oxaliplatin compositions according to example 1 and example 2 (sparged composition according to present invention) showed increased stability as compared to oxaliplatin composition according to example 3 (non-sparged composition). Sparged aqueous oxaliplatin composition maintains the pH of the composition in between 3 to 4.5, which in turn helps in reducing the hydrolysis of oxaliplatin present in the composition and hence reduces the formation of Impurities A and Impurities B of oxaliplatin in the composition. Further, sparging of carbon dioxide in the oxaliplatin composition helps in replacing the dissolved oxygen from the oxaliplatin composition by carbon dioxide, which in turn reduces the oxidation of oxaliplatin and formation of Impurity C of oxaliplatin in the composition.

1. A stable parenteral composition of oxaliplatin having pH between 3 to 4.5 comprises of a solution of oxaliplatin in water wherein the said pH is attained by sparging of carbon dioxide in the composition.

2. A composition according to claim 1, wherein the concentration of oxaliplatin is in a range of 1 mg/ml to 7 mg/ml.

3. A composition according to claim 1, which optionally comprises of pharmaceutically acceptable carbohydrates selected from lactose mono hydrate, glucose, maltose, fructose, trehalose, sucrose, galactose, dextran or mixtures thereof.

4. A composition according to claim 3, wherein the concentration of pharmaceutically acceptable carbohydrates is in a range of 10 mg/ml to 70 mg/ml.

5. A composition according to claim 1, wherein the oxaliplatin content in the composition will not reduce less than 98% of the initial oxaliplatin concentration and the solution remains clear, colorless and free from particulates after storage for a pharmaceutically acceptable duration.

6. A stable parenteral composition of oxaliplatin having pH between 3 to 4.5, comprises of 5 mg/ml of oxaliplatin dissolved in water, 45 mg/ml of pharmaceutically acceptable carbohydrates selected from lactose mono hydrate, glucose, maltose, fructose, trehalose, sucrose, galactose, dextran or mixtures thereof, wherein the said pH is attained by sparging of carbon dioxide in the composition.

7. A stable parenteral composition of oxaliplatin having pH in between 3 to 4.5 comprises of 5 mg/ml of oxaliplatin dissolved in water, wherein the said pH is attained by sparging of carbon dioxide in the composition.

8. A process for the preparation of a stable parenteral composition of oxaliplatin having pH in between 3 to 4.5 comprises the steps of dissolving oxaliplatin in water to obtain a clear solution, optionally dissolving pharmaceutically acceptable carbohydrates and sparging the obtained solution with carbon dioxide till the said pH is attained.

9. A stable parenteral composition of oxaliplatin in aqueous solution having pH between 3 to 4.5 attained by sparging carbon dioxide in the composition.
